(19) United States
(12) Patent Application Publication
(10) Pub. No.: US 2016/0082057 A1
(43) Pub. Date: Mar. 24, 2016

(54) METHODS AND FORMULATIONS FOR REDUCING ABSORPTION OF CARBOHYDRATES IN A COMPANION ANIMAL

(71) Applicant: NESTEC SA, Vevey (CH)

(72) Inventor: Yuanlong Pan, Chesterfield, MO (US)

(21) Appl. No.: 14/857,089

(22) Filed: Sep. 17, 2015

Related U.S. Application Data
(60) Provisional application No. 62/052,584, filed on Sep. 19, 2014.
(60) Provisional application No. 62/052,580, filed on Sep. 19, 2014.

(51) Int. Cl. A61K 36/02; A61K 47/36; A61K 47/26; A23K 1/16; A61K 47/14; A61K 9/00; G01N 33/66; A23K 1/18; A61K 36/48; A61K 47/42

(52) U.S. Cl. CPC A61K 36/02; A61K 36/48; A61K 47/36; A61K 47/26; A61K 47/42; A61K 47/14; A61K 9/009; A61K 9/0056; G01N 33/66; A23K 1/1846; A23K 1/1631; A23K 1/164; A23K 1/1643; A23K 1/1646

(57) ABSTRACT

The invention provides methods for reducing absorption of carbohydrates in a companion animal, comprising identifying the companion animal having a health condition or at risk for the health condition associated with high carbohydrate ingestion and feeding a dietary formulation in a therapeutically effective amount to the companion animal. Such dietary formulations can be any of those as described herein. Generally, the dietary formulation can comprise 20% to 60% protein, 10% to 40% fat, 10% to 50% carbohydrates, and 0.01% to 5% alpha-amylase inhibitor. Typically, the therapeutically effective amount can be effective for reducing absorption of carbohydrates in the companion animal as measured by lowering the postprandial blood glucose of the animal as compared to the postprandial blood glucose of the companion animal ingesting a comparable dietary formulation that excludes the alpha-amylase inhibitor.

### CROSS REFERENCE TO RELATED APPLICATIONS

This application claims priority to U.S. Provisional Application Ser. No. 62/052,584 filed Sep. 19, 2014 and U.S. Provisional Application Ser. No. 62/052,580 filed Sep. 19, 2014, the disclosures of both of which are incorporated in their entireties herein by this reference.

### BACKGROUND OF THE INVENTION

1. Field of the Invention

The invention relates generally to methods and compositions for the reducing absorption of carbohydrates in a companion animal and for reducing postprandial blood glucose in a companion animal.

2. Description of Related Art

Energy in the body is generated by the production of ATP from “food stuff.” More specifically, when food is consumed it is broken down into its constituent parts consisting primarily of simple and complex carbohydrates, fats, proteins and indigestible fiber such as cellulose. The carbohydrates, fats and proteins are then further broken down into their basic units: carbohydrates into simple sugars, proteins into amino acids and small peptides, and fats into fatty acids and glycerol. The body then uses these basic units to generate substances it needs for growth, maintenance and energy production. Carbohydrates, proteins and fats can all be metabolized to provide energy in the form of ATP, however, carbohydrates or fats are the primary substrates used by the body for the generation of ATP via glycolysis and the Kreb's cycle, depending on the dietary compositions.

Depending on the size of the molecule carbohydrates are classified as either simple or complex. Simple carbohydrates are small molecules, specifically mono- and disaccharides, such as glucose, fructose, galactose, and sucrose. Complex carbohydrates or polysaccharides are comprised of long chains of simple carbohydrates. The most important polysaccharides are starch, glycogen and cellulose, which are all polymers of glucose differing only in the way the glucose molecules are linked. Glycogen is the energy reservoir in animals, starch is the energy reservoir in plants and cellulose is the major structural component of plants. While most forms of starch are digestible, humans lack the enzyme necessary to digest cellulose and therefore it becomes part of our dietary fiber.

Over half of the carbohydrates consumed by humans have traditionally come from starch sources such as breads and grains. Starch is a mixture of amylose and amylopectin. Amylose is a linear polysaccharide consisting of glucose molecules covalently bonded by α-1,4 linkages. Amylopectin is a branched polysaccharide consisting of glucose molecules covalently bonded with one α-1,6 linkage per approximately thirty α-1,4 linkages. Starch is rapidly hydrolyzed by α-amylase, which is secreted by the salivary glands and the pancreas. Upon hydrolysis, amylose is broken down into small straight chain oligosaccharides, such as maltose (two glucose molecules in a α-1,4 linkage) and maltotriose (three glucose molecules in α-1,4 linkages). Amylopectin is broken down into small straight chain oligosaccharides, as well as, into the branched oligosaccharide α-dextrin (several glucose molecules linked by both α-1,4 linkages and α-1,6 linkages). These sugars are further broken down into glucose monomers by the enzymes maltase and β-amylase.

Other carbohydrates consumed in our diet are simple carbohydrates, such as the monosaccharides glucose and fructose and the disaccharide sucrose. Glucose is present in low levels in most natural foods, whereas fructose is obtained primarily from processed foods, sweeteners and to a minor extent from fruits and certain vegetables. Fructose is produced synthetically via the enzymatic isomerization of dextrose. (Bhosale et al. (1996) Microbiol. Rev. 60:280-300). Sucrose, another well-known sweetener, is comprised of a glucose and a fructose in an α-1,2 linkage between C1 of glucose and C2 of fructose. Sucrose is hydrolyzed by the enzyme sucrase in the intestinal mucosa to provide glucose and fructose. (Dahlqvist (1972) Acta Med. Scand. Suppl. 542:13-18).

Of all the problems associated with high fructose intake, the effect fructose has on lipid accumulation, lipogenesis and weight gain is especially significant. In developed countries, low-fat, high-carbohydrate diets are prevalent owing to the extreme awareness regarding the dangers of fat and cholesterol in cardiovascular disease, endorsement of fructose as a substitute sweetener for diabetics and a lack of awareness of the dangers of fructose consumption. (Gerrits and Tsalikian (1993) Am. J. Clin. Nutr. 58(Supp.):7965-799S; Sonko et al. (1993) Acta Physiol. Scand. 147:99-108). This has led to a tremendous increase in carbohydrates including fructose consumption correlating with increases in weight gain and obesity over the last thirty years.

Generally, high carbohydrate intake has been related to higher risk of numerous diseases including coronary heart disease (Liu et at Am J Clin Nutr 2000;71:1455-61.), metabolic syndrome (McKeown et al., Diabetes Care 27:538-546, 2004), type II diabetes (Salmerson et al. JAMA 277:472-477, 1997), obesity (Roberts, Nutr Rev 58:163-169, 2000), age-related macular degeneration (Chiu et al., Am J Clin Nutr 2007;86:1210-8), Alzheimer's disease (Seneff et al. European Journal of Internal Medicine, 22:134-140, 2011), and cancer (Michaud et al. J Natl Cancer Inst, 94:1293-1300, 2002; Romieu et al., Cancer Epidemiol Biomarkers Prey, 13:1283-1289, 2004; Michaud et al., Cancer Epidemiol Biomarkers Prey, 14:138-143,2005; Silvera et al., Int. J. Cancer: 114:653-658, 2005.). As such, research and development efforts for maintaining healthy levels of carbohydrates continue.

### SUMMARY OF THE INVENTION

It is, therefore, an object of the invention to provide methods for reducing absorption of carbohydrates in a companion animal. In one embodiment, a method for reducing absorption of carbohydrates in a companion animal can comprise identifying a companion animal having a health condition or at risk for a health condition associated with high carbohydrate ingestion and feeding a dietary formulation in a therapeutically effective amount to the companion animal, wherein the therapeutically effective amount is effective for reducing absorption of carbohydrates in the companion animal as measured by lowering the postprandial blood glucose of the animal as compared to the postprandial blood glucose of the companion animal ingesting a comparable dietary formulation that excludes the alpha-amylase inhibitor. In some embodiments, the dietary formulations can be food compositions.

It is another object of the invention to provide methods for reducing postprandial blood glucose in a companion animal. In one embodiment, the method can comprise feeding a dietary formulation in a therapeutically effective amount to the companion animal, where the therapeutically effective amount is effective for reducing the postprandial blood glucose by at least 10% after 1 hour of the feeding of the companion animal as compared to the postprandial blood glucose after 1 hour of the feeding of the companion animal a comparable dietary formulation that excludes the alpha-amylase inhibitor.

It is another object of the invention to provide dietary formulations for reducing absorption of carbohydrates in a companion animal or for reducing the postprandial blood glucose in a companion animal. In one embodiment, the dietary formulation can comprise 20% to 60% protein, 10% to 40% fat, 10% to 50% carbohydrates, 1% to 50% resistant starch, and 0.01% to 5% alpha-amylase inhibitor.

Other and further objects, features, and advantages of the present invention will be readily apparent to those skilled in the art.

### DETAILED DESCRIPTION OF THE INVENTION

### Definitions

The term “companion animal” means domesticated animals such as cats, dogs, rabbits, guinea pigs, ferrets, hamsters, mice, gerbils, horses, cows, goats, sheep, donkeys, pigs, and the like. In one aspect, companion animal can be a dog and/or cat.

The term “alpha-amylase inhibitor” refers to any extract or composition that exhibits alpha-amylase inhibitor activity, e.g., StarchLite®. In one aspect, the alpha-amylase inhibitor can refer to any protein that complexes with alpha-amylase.

The term “resistant starch” refers to starches and starch degradation products that resist digestion and passes through to the large intestine of an animal where it acts like dietary fiber including: those that are physically inaccessible or digestible resistant starch, such as that found in seeds or legumes and unprocessed whole grains; those that occur in natural granular form, such as uncooked potato, green banana and high amylose corn; those that are formed when starch-containing foods are cooked and cooled such as in legumes, bread, cornflakes and cooked-and-chilled potatoes, pasta salad or sushi rice, due to retrogradation, which refers to the collective processes of dissolved starch becoming less soluble after being heated and dissolved in water and then cooled; and those that have been chemically modified to resist digestion.

The term “therapeutically effective amount” means an amount of a compound of the invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “comparable dietary formulation” means a dietary formulation that is the same as those described herein except that the comparable dietary formulation excludes alpha-amylase inhibitors. In all other aspects, the comparable dietary formulation is the same as that of the dietary formulations disclosed herein, including containing the same ingredients with the same ratios of ingredients.

The term “complete and balanced” when referring to a food composition means a food composition that contains all known required nutrients in appropriate amounts and proportions based on recommendations of recognized authorities in the field of animal nutrition, and are therefore capable of serving as a sole source of dietary intake to maintain life or promote production, without the addition of supplemental nutritional sources. Nutritionally balanced pet food and animal food compositions are widely known and widely used in the art, e.g., complete and balanced food compositions formulated according to standards established by the Association of American Feed Control Officials (AAFCO).

The term “single package” means that the components of a kit are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof. A single package may be containers of individual dietary compositions of the invention physically associated such that they are considered a unit for manufacture, distribution, sale, or use.

The term “virtual package” means that the components of a kit are associated by directions on one or more physical or virtual kit components instructing the user how to obtain the other components, e.g., a bag or other container containing one component and directions instructing the user to go to a website, contact a recorded message or a fax-back service, view a visual message, or contact a caregiver or instructor to obtain instructions on how to use the kit or safety or technical information about one or more components of a kit.

The term “about” means plus or minus 20%; in one aspect, plus or minus 10%; in another aspect, plus or minus 5%; and in one specific aspect, plus or minus 2%.

All percentages expressed herein are by weight or amount of the total weight or amount of the composition unless expressed otherwise.

The invention is not limited to the particular methodology, protocols, and reagents described herein because they may vary. Further, the terminology used herein is for the purpose of describing particular embodiments only and is not intended to limit the scope of the present invention.

As used herein, the singular form of a word includes the plural, and vice versa, unless the context clearly dictates otherwise. Thus, the references “a”, “an”, and “the” are generally inclusive of the plurals of the respective terms. Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

Unless defined otherwise, all technical and scientific terms and any acronyms used herein have the same meanings as commonly understood by one of ordinary skill in the art in the field of the invention. Although any compositions, methods, articles of manufacture, or other means or materials similar or equivalent to those described herein can be used in the practice of the present invention, the preferred compositions, methods, articles of manufacture, or other means or materials are described herein.

As used herein, embodiments, aspects, and examples using “comprising” or other open ended language can be substituted with “consisting essentially of” and “consisting of” embodiments.

As used throughout, ranges are used herein as shorthand, so as to avoid having to set out at length and describe each and every value within the range. Any appropriate value within the range can be selected, where appropriate, as the upper value, lower value, or the terminus of the range. It is understood that any and all whole or partial integers between any ranges or intervals set forth herein are included herein.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, are relevant prior art for the present invention and the right to challenge the accuracy and pertinence of such patents, patent applications, publications, and other references is specifically reserved.

### The Invention

Elevated carbohydrate intake is associated with a number of health conditions including coronary heart disease, metabolic syndrome, type II diabetes, obesity, age-related macular degeneration, Alzheimer's disease, and cancer. As such, the present inventors have discovered that using amylase inhibitors over other types of inhibitors in dietary formulations for companion animals can more effectively reduce dietary carbohydrate digestion and absorption in a normal protein diet. Such dietary formulation can provide the benefits of low carbohydrate diets but avoid problems common to such diets.

In light of these discoveries, a method for reducing absorption of carbohydrates in a companion animal can comprise identifying the companion animal having a health condition or at risk for the health condition associated with high carbohydrate ingestion and feeding a dietary formulation in a therapeutically effective amount to the companion animal. Such dietary formulations can be any of those as described herein. In one embodiment, the dietary formulation can be formulated as a pet food composition. Additionally, the present disclosure includes the present dietary formulations for use in the treatment of a health condition including coronary heart disease, metabolic syndrome, type II diabetes, obesity, age-related macular degeneration, Alzheimer's disease, and cancer.

Dietary formulations of the invention can be administered to the animal in any suitable form using any suitable administration route. For example, the dietary formulations can be administered in a dietary formulation composition, in a food composition, in a dietary supplement, in a pharmaceutical composition, in a nutraceutical composition, or as a medicament. Similarly, the dietary formulations can be administered using a variety of administration routes, including oral, intranasal, intravenous, intramuscular, intragastric, transpyloric, subcutaneous, rectal, and the like. In one embodiment, the dietary formulations are administered to an animal orally. In one aspect, the dietary formulations can be administered orally to an animal as a dietary supplement, as a food composition, or as an ingredient in a food composition.

In a one embodiment, the dietary formulations can be administered to an animal as an ingredient in a food composition suitable for consumption by an animal, including companion animals such as dogs and cats. Such compositions include complete foods intended to supply the necessary dietary requirements for an animal or food supplements such as animal treats.

Generally, the dietary formulation can comprise 20% to 60% protein, 10% to 40% fat, 10% to 50% carbohydrates, and 0.01% to 5% alpha-amylase inhibitor. In one embodiment, the dietary formulation can further comprise resistant starch. In one aspect, the resistant starch can be present in the dietary formulation an amount of 1% to 50%. In another aspect, the resistant starch can be present in the dietary formulation an amount of 5% to 40%. Typically, the therapeutically effective amount can be effective for reducing absorption of carbohydrates in the companion animal as measured by lowering the postprandial blood glucose of the animal as compared to the postprandial blood glucose of the companion animal ingesting a comparable dietary formulation that excludes the alpha-amylase inhibitor, excludes the resistant starch, or a dietary formulation that includes a different inhibitor.

As discussed herein, the present methods are generally used with companion animals. In one aspect, the companion animal can be a dog. In another aspect, the companion animal can be a cat.

In one embodiment, the dietary formulation can be formulated as a pet food composition. In one aspect, such dietary formulations can include has 20% to 60% protein, 10% to 40% fat, 10% to 50% carbohydrates, and 0.01% to 5% alpha-amylase inhibitor. In another embodiment, the dietary formulations can include has 25% to 55% protein, 15% to 35% fat, 15% to 45% carbohydrates, and 0.05% to 1% alpha-amylase inhibitor. Other components that can be present include probiotics, dietary fibers, omega-3 polyunsaturated fatty acids, monounsaturated fatty acids, antioxidants, medium chain triglycerides, inhibitors for hexokinase and glucokinase. In another embodiment, the alpha-amylase inhibitors can be incorporated into a pet food from a sachet or can supplement a pet food by being incorporated into a treat.

The moisture content for such food compositions varies depending on the nature of the food composition. The food compositions may be dry compositions (e.g., kibble), semi-moist compositions, wet compositions, or any mixture thereof. In one embodiment, the composition can be a complete and nutritionally balanced pet food. In this embodiment, the pet food may be a “wet food”, “dry food”, or food of “intermediate moisture” content. “Wet food” describes pet food that is typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one embodiment, the compositions have moisture content from about 5% to about 20%. Dry food products include a variety of foods of various moisture contents, such that they are relatively shelf-stable and resistant to microbial or fungal deterioration or contamination. Other food compositions include dry food compositions that are extruded food products such as pet foods or snack foods for companion animals.

In another embodiment, the dietary formulations can be administered to an animal in a dietary supplement. The dietary supplement can have any suitable form such as a gravy, drinking water, beverage, yogurt, powder, granule, paste, suspension, chew, morsel, treat, snack, pellet, pill, capsule, tablet, sachet, or any other suitable delivery form. The dietary supplement can comprise the dietary formulations and optional compounds such as vitamins, preservatives, probiotics, prebiotics, and antioxidants. This permits the supplement to be administered to the animal in small amounts, or in the alternative, can be diluted before administration to an animal. The dietary supplement may require admixing with a food composition or with water or other diluent prior to administration to the animal. When administered in a dietary supplement, the dietary formulations comprise from about 0.1 to about 90% of the supplement, from about 3 to about 70%, or even from about 5 to about 60%.

In another embodiment, the dietary formulations can be administered to an animal in a pharmaceutical or nutraceutical composition. The pharmaceutical composition can comprise the dietary formulations and one or more pharmaceutically or nutraceutically acceptable carriers, diluents, or excipients. Generally, pharmaceutical compositions are prepared by admixing a compound or composition with excipients, buffers, binders, plasticizers, colorants, diluents, compressing agents, lubricants, flavorants, moistening agents, and the like, including other ingredients known to skilled artisans to be useful for producing pharmaceuticals and formulating compositions that are suitable for administration to an animal as pharmaceuticals. When administered in a pharmaceutical or nutraceutical composition, the dietary formulations comprise from about 0.1 to about 90% of the composition, preferably from about 3 to about 70%, more preferably from about 5 to about 60%.

The dietary formulations of the invention can be administered to the animal on an as-needed, on an as-desired basis, or on a regular basis. A goal of administration on a regular basis is to provide the animal with a regular and consistent dose of the dietary formulations or the direct or indirect metabolites that result from such ingestion. Such regular and consistent dosing will tend to create constant blood levels of the dietary formulations and their direct or indirect metabolites. Thus, administration on a regular basis can be once monthly, once weekly, once daily, or more than once daily. Similarly, administration can be every other day, week, or month, every third day, week, or month, every fourth day, week, or month, and the like. Administration can be multiple times per day. When utilized as a supplement to ordinary dietetic requirements, the dietary formulations may be administered directly to the animal, e.g., orally or otherwise. The dietary formulations can alternatively be contacted with, or admixed with, daily feed or food, including a fluid, such as drinking water, or an intravenous connection for an animal that is receiving such treatment. Administration can also be carried out as part of a dietary regimen for an animal. For example, a dietary regimen may comprise causing the regular ingestion by the animal of the dietary formulations in an amount effective to accomplish the methods of the invention.

According to the methods of the invention, administration of the dietary formulations, including administration as part of a dietary regimen, can span a period ranging from parturition through the adult life of the animal. In various embodiments, the animal can be a companion animal such as a dog or cat. In certain embodiments, the animal can be a young or growing animal. In more other embodiments, the animal can be an aging animal. In other embodiments administration begins, for example, on a regular or extended regular basis, when the animal has reached more than about 30%, 40%, or 50% of its projected or anticipated lifespan. In some embodiments, the animal has attained 40, 45, or 50% of its anticipated lifespan. In yet other embodiments, the animal can be older having reached 60, 66, 70, 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan.

The dietary formulations of the invention can be administered to an animal for a time required to accomplish one or more objectives of the invention, e.g., the treatment of cancer, reduction of cancer risk, or prevention of cancer in a companion animal or reducing postprandial blood glucose. In one embodiment, the dietary formulations can be administered to an animal on a regular basis.

Regarding the present methods, such methods general include a therapeutically effective amount of a dietary formulation. In one embodiment, the dietary formulation can be administered in a therapeutically effective amount ranging from 0.1 mg/kg per body weight (BW) of the animal to 500 mg/kg BW of the alpha-amylase inhibitors and/or 0.13 g/kg BW to 7.0 g/kg BW resistant starch. In one aspect, the therapeutically effective amount can be from 1 mg/kg BW to 50 mg/kg BW of the alpha-amylase inhibitors and/or 0.67 g/kg BW to 5.0 g/kg BW resistant starch. In another aspect, the therapeutically effective amount can be from 10 mg/kg BW to 40 mg/kg BW of the alpha-amylase inhibitors and/or 1.3 g/kg BW to 4.0 g/kg BW resistant starch.

In another embodiment, a method for reducing postprandial blood glucose in a companion animal can comprise feeding a dietary formulation in a therapeutically effective amount to the companion animal, the dietary formulation being any of those described herein. Generally, the therapeutically effective amount is effective for reducing the postprandial blood glucose by at least 10% after 1 hour of the feeding of the companion animal as compared to the postprandial blood glucose after 1 hour of the feeding of the companion animal a comparable dietary formulation that excludes the alpha-amylase inhibitor.

In one embodiment, the postprandial blood glucose can be at least 15% lower after 1 hour of feeding compared to the postprandial blood glucose of the companion animal when fed the comparable food. In some aspects, the postprandial blood glucose can be at least 10% lower, at least 8% lower, or at least 5% lower after 1 hour. In other aspects, the postprandial blood glucose can be at least 15% lower, at least 10% lower, or at least 8% lower after 2 hours, or even at least 5% lower, or at least 3% lower after 2 hours. In still other aspects, the postprandial blood glucose can be at least 8% lower, at least 5% lower, or at least 3% lower after 3 hours.

In a further aspect, the invention provides kits suitable for implementing the methods of the invention. The kits comprise in separate containers in a single package or in separate containers in a virtual package, as appropriate for the kit component, (1) a dietary formulation as described herein; and (2) instructions for using the dietary formulations for treatment of a companion animal having a health condition or at risk for the health condition associated with high carbohydrate ingestion by lowering the postprandial blood glucose of the animal as compared to the postprandial blood glucose of the animal ingesting a comparable dietary formulation that excludes the alpha-amylase inhibitor and/or the resistant starch. In certain embodiments, the kits further comprise one or more of one or more alpha-amylase inhibitors in a separate container or sachet.

When the kit comprises a virtual package, the kit is limited to instructions in a virtual environment in combination with one or more physical kit components. Generally, the kit contains the dietary formulations and other physical components in amounts sufficient to implement the methods of the invention and the virtual package contains the instructions relating to using the physical components to implement the methods of the invention.

In another aspect, the invention provides a means for communicating information about or instructions for treatment of a companion animal having a health condition or at risk for the health condition associated with high carbohydrate ingestion by lowering the postprandial blood glucose of the animal. The means comprises a document, digital storage media, optical storage media, audio presentation, or visual display containing the information or instructions. In certain embodiments, the communication means is a displayed web site, visual display, brochure, product label, package insert, advertisement, handout, public announcement, audiotape, videotape, DVD, CD-ROM, computer readable chip, computer readable card, computer readable disk, computer memory, or combination thereof containing such information or instructions.

Useful information includes one or more of (1) recommended feeding schedules for the animal, particularly based on the animal's species and health condition (e.g., type of cancer), (2) recommended health promoting agents to be administered in conjunction with the use of the recommended feeding pattern, and (3) contact information for animals or their caregivers to use if they have a question about the invention and its use.

### EXAMPLES

The invention can be further illustrated by the following examples, although it will be understood that these examples are included merely for purposes of illustration and are not intended to limit the scope of the invention unless otherwise specifically indicated.

### Example 1

### Postprandial Blood Glucose Study Using Alpha Amylase

Eight adult dogs were recruited in the study. The dogs were fed to meet their maintenance energy requirement during the study.

### Diets, Feeding Protocol, and Sample Collection

The control dry dog food contained 25% protein, 13% fat and 48% carbohydrates. The test diets were based on the control formula supplemented with either 0.1% StarchLite® from Ingredia Nutritional (a white bean extract with alpha-amylase inhibitor activity) or InSea2® from innoVactiv inc. (a polyphenol extract from seaweeds with both alpha-amylase and alpha-glucosidase inhibitor activity). The dogs were fed the control diet for 7 days and blood samples were collected at 15-minute interval for 3 hours after the dog ate the diet on day 7. After one-week wash-out phase, the dogs were fed the InSea2® diet for 7 days. On day 7, blood samples were collected at 15-minute interval for 3 hours after the dog ate the diet. Blood samples were analyzed for glucose concentrations, and there were 4 sample collections per hour. After one-week wash-out phase, the dogs were fed the StarchLite® diet for 7 days. On day 7, blood samples were collected at 15-minute interval for 3 hours after the dog ate the diet. Blood samples were analyzed for glucose concentrations, and there were 4 sample collections per hour

### Results

The average postprandial blood glucose for each hour was calculated based on the four blood glucose values and summarized in following table. The data indicate that the

StarchLite® significantly reduced postprandial blood glucose without reduction of dietary carbohydrates. Surprisingly, InSea2® didn't reduce postprandial blood glucose without reduction of dietary carbohydrates

TABLE 1
Hourly average postprandial blood glucose

| | Average postprandial blood glucose | | |
|---|---|---|---|
| | (mg/dL, mean ± SEM) | | |
| Composition | First Hour* | Second Hour* | Third Hour* |
| Control diet | 87.7 ± 1.5 | 91.0 ± 1.6 | 90.8 ± 1.3 |
| alpha-amylase inhibitor | 74.6 ± 1.5 | 83.2 ± 1.6 | 82.7 ± 1.3 |
| alpha-amylase/ | 96.5 ± 1.5 | 93.9 ± 1.6 | 96.8 ± 1.3 |
| alpha-glucosidase inhibitor | | | |

*Values are average of 4 data points taken at 15 minute intervals during each hour after administration

TABLE 2
Average postprandial blood glucose
(mg/dl) within 3 hours after a meal

| | | 15 | 30 | 45 | 60 | 75 | 90 |
|---|---|---|---|---|---|---|---|
| Composition | 0 | min | min | min | min | min | min |
| Control | 76.0 | 81.9 | 89.6 | 89.1 | 90.3 | 91.5 | 91.2 |
| alpha-amylase | 72.9 | 72.1 | 75.9 | 76.1 | 74.4 | 84.7 | 87.1 |
| inhibitor | | | | | | | |
| alpha-amylase/ | 86.3 | 85.2 | 94.5 | 103.1 | 103.5 | 98.8 | 92.4 |
| alpha-glucosidase | | | | | | | |
| inhibitor | | | | | | | |
| | 105 | 120 | 135 | 150 | 165 | 180 | |
| | min | min | min | min | min | min | — |
| Control | 89.6 | 91.9 | 93.5 | 89.8 | 94.7 | 96.6 | — |
| alpha-amylase | 86.0 | 75.2 | 77.7 | 83.6 | 80.3 | 89.1 | — |
| inhibitor | | | | | | | |
| alpha-amylase/ | 90.3 | 94.2 | 94.1 | 94.9 | 98.6 | 99.7 | — |
| alpha-glucosidase | | | | | | | |
| inhibitor | | | | | | | |

The data in Table 2 showed that the alpha-amylase inhibitor was effective in reducing postprandial blood glucose without limiting dietary carbohydrates for 3 hours after feeding, but the alpha-amylase/alpha-glucosidase inhibitor combination failed to prevent the increase in postprandial blood glucose. Such findings are particularly unexpected as InSea2® has been shown to be effective in human trials to reduce post-meal blood glucose response (“A Randomised Crossover Placebo-Controlled Trial Investigating the Effect of Brown Seaweed (Ascophyllum nodosum and Fucus vesiculosus) on Postchallenge Plasma Glucose and Insulin Levels in Men and Women” by Paradis et al., Applied Physiology, Nutrition, and Metabolism, 36(6): 913-919, (2011); see also (http://insea2.com/the-solution/what-insea2-is/).

### Example 2

### Postprandial Blood Glucose Study Using Alpha-Amylase and Resistant Starch

Adult dogs were recruited in the study. The dogs were fed to meet their maintenance energy requirement during the study.

### Diets, Feeding Protocol, and Sample Collection

The control dry dog food contained 21% protein, 10% fat, 10% fiber, 46% carbohydrates, and 12% moisture. The test diets were based on the control formula supplemented with either 0.1% StarchLite® from Ingredia Nutritional (a white bean extract with alpha-amylase inhibitor activity) and/or 30% resistant starch according to the schedule listed below.

There was a one-week wash-out period between each diet, and only the Control diet was fed during the wash-out period. First, the dogs were fed the Control diet for 7-days, and blood samples were collected at 90 and 105 minutes after meal on Day 7. Blood draw was done with a cephalic catheter. The blood samples were subject to blood glucose measurements. After a wash-out period, the dogs were switched to the control diet supplemented with 0.1% StarchLite® for 7 days, blood samples were collected at 90 and 105 minutes after meal at the end of the feeding (Day 7). The blood samples were analyzed for glucose levels. After a wash-out period, the dogs were switched to the control diet supplemented with 30% resistant starch for 7 days, blood samples were collected at 90, and 105 minutes after meal at the end of the feeding (Day 7). The blood samples were analyzed for glucose levels. After a wash-out period, the dogs were switched to the control diet supplemented with 0.1% StarchLite® and 30% resistant starch for 7 days, blood samples were collected at 90, and 105 minutes after meal at the end of the feeding (Day 7). The blood samples were analyzed for glucose levels.

### Results

The average postprandial blood glucose was calculated and summarized in following table. The data indicate that the combination of alpha-amylase inhibitor with resistant starch unexpected provided a decrease in postprandial blood glucose as compared to either component alone.

TABLE 3
Postprandial blood glucose (mg/dl) after a meal

| Composition | 90 min | 105 min |
|---|---|---|
| Control | 100 | 96.3 |
| alpha-amylase inhibitor | 99.65 | 97.45 |
| resistant starch | 98.6 | 95.87 |
| resistant starch + alpha-amylase inhibitor | 93.55 | 92.55 |

The data in Table 3 showed that, while the alpha-amylase inhibitor and resistant starch generally showed slight improvement in reducing postprandial blood glucose without limiting dietary carbohydrates after feeding, the combination of resistant starch and alpha-amylase showed a more than additive effect in lowering postprandial blood glucose after about 1.5 hours after feeding. Such findings are unexpected.

In the specification, there have been disclosed typical embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

What is claimed is:

1. A method for reducing absorption of carbohydrates in a companion animal, the method comprising:
identifying the companion animal having a health condition or at risk for the health condition associated with high carbohydrate ingestion; and
feeding a dietary formulation in a therapeutically effective amount to the companion animal, the dietary formulation comprising:
20% to 60% protein;
10% to 40% fat;
10% to 50% carbohydrates; and
0.01% to 5% alpha-amylase inhibitor;
wherein the therapeutically effective amount is effective for reducing absorption of carbohydrates in the companion animal as measured by lowering the postprandial blood glucose of the animal as compared to the postprandial blood glucose of the companion animal ingesting a comparable dietary formulation that excludes the alpha-amylase inhibitor.

2. The method of claim 1, further comprising applying the alpha-amylase inhibitor to the dietary formulation from a sachet.

3. The method of claim 1, wherein the health condition is selected from the group consisting of coronary heart disease, metabolic syndrome, type II diabetes, obesity, age-related macular degeneration, Alzheimer's disease, and cancer.

4. The method of claim 1, wherein the dietary formulation is formulated as a pet food composition.

5. The method of claim 1, wherein the companion animal is a dog.

6. The method of claim 1, wherein the therapeutically effective amount includes from 0.1 mg/kg BW to 500 mg/kg BW of the alpha-amylase inhibitor.

7. The method of claim 1, wherein the dietary formulation further comprises resistant starch.

8. The method of claim 7, wherein the dietary formulation has 25% to 55% protein, 15% to 35% fat, 15% to 45% carbohydrates, 1% to 50% resistant starch, and 0.05% to 1% alpha-amylase inhibitor.

9. The method of claim 1, wherein the postprandial blood glucose is at least 15% lower after 1 hour of feeding compared to the postprandial blood glucose of the companion animal when fed the comparable food.

10. A dietary formulation for reducing absorption of carbohydrates in a companion animal, the composition comprising:
20% to 60% protein;
10% to 40% fat;
10% to 50% carbohydrates; and
0.01% to 5% alpha-amylase inhibitor.

11. The dietary formulation of claim 10, wherein the dietary formulation further comprises 1% to 50% resistant starch.

12. The dietary formulation of claim 11, wherein the dietary formulation has 25% to 55% protein, 15% to 35% fat, 15% to 45% carbohydrates, 5% to 40% resistant starch, and 0.05% to 1% alpha-amylase inhibitor.

13. The dietary formulation of claim 10, wherein the dietary formulation is formulated as a dry extruded food composition.

14. The dietary formulation of claim 10, wherein the dietary formulation is formulated as a semi-moisture pet food composition.

15. The dietary formulation of claim 10, wherein the dietary formulation is formulated as a complete and balanced pet food composition.

16. The dietary formulation of claim 10, wherein the alpha-amylase inhibitor is a powder that is added to the dietary formulation subsequent extrusion or mixing of the protein, fat, and carbohydrates.
